(19) United States
(12) Patent Application Publication
(10) Pub. No.: US 2019/0254901 A1
(43) Pub. Date: Aug. 22, 2019

(54) Infant Care Apparatus and System

(71) Applicants: Glenn Fernandes, Thane (IN); Sarita Parikh, Mumbai (IN)

(72) Inventors: Glenn Fernandes, Thane (IN); Sarita Parikh, Mumbai (IN)

(21) Appl. No.: 16/341,982

(22) PCT Filed: Nov. 12, 2017
(86) PCT No.: PCT/IN2017/050526

§ 371 Date: Apr. 15, 2019

(30) Foreign Application Priority Data
Nov. 14, 2016 (IN) 201621038740

(51) Int. Cl. A61G 11/00; A61F 13/42; A61F 5/44; A61G 10/02; A61H 31/02; A61N 5/06; A61M 1/00; A61F 7/00; A61M 21/02; A61B 5/0478; A61B 5/01; A61B 5/024; A47D 13/02

(52) U.S. Cl. CPC A61G 11/00; A61M 2021/0027; A61F 5/4407; A61G 10/02; A61H 31/02; A61N 5/0616; A61M 1/0023; A61F 7/0053; A61F 7/0085; A61M 21/02; A61B 5/0478; A61B 5/01; A61B 5/02438; A47D 13/02; A61F 2005/4402; A61N 2005/0652; A61N 2005/0636; A61F 2007/0062; A61F 13/42

(57) ABSTRACT

Infant care apparatus and system comprising a tray molded in the shape of an infant's body; and fastening devices to fasten the tray around the caregiver's chest, to provide safe kangaroo care to infants. Some embodiments may include sensors to monitor the infant's physiological parameters, and/or a negative pressure ventilator and/or an automated urine and feces collection and disposal system, and/or an external cardiac compression system, and/or electroencephalograph cap, and/or a heat pump to maintain infant body temperature; and/or light emitting diodes to provide phototherapy; and/or an aspirator for suctioning secretions from nose and/or mouth of infant; and/or warm mist generating apparatus to humidify air around the infant. It may include a networking module for transmission of sensor data to and receiving of operating commands from remote servers accessible by medical professionals. The invention thus also provides safe and easy-to-operate intensive care for infants, along with kangaroo care.

### CROSS-REFERENCE TO RELATED APPLICATIONS

This application claims the benefit of PCT Application No. PCT/IN2017/050526 filed on Nov. 12, 2017.

### FIELD OF INVENTION

This invention relates to the field of infant care; specifically to apparatuses and systems to handle and manage infants, and to monitor and stabilize the physiological parameters of infants.

### BACKGROUND OF INVENTION

Infant morbidity and mortality rates are very high across the world, despite technological progress.

Life-saving intensive care equipment for infants is very expensive, complex and requires highly skilled staff to operate. The acute scarcity of such equipment and skilled staff leads to unnecessary infant deaths, especially in developing countries. Preterm infants suffer the most due to lack of suitable intensive care availability, and more than 10% births worldwide are preterm.

Even developed countries, with sufficient infant intensive care equipment, are plagued by extreme shortage of skilled nursing staff. Infants requiring neonatal intensive care need at least one skilled staff per infant, which is rarely possible. Further, the medical equipment available for infants is not very safe. E.g. the conventional positive pressure ventilators can damage the delicate lungs of preterm infants and lead to chronic lung disease of the newborn, pneumonia and even brain damage.

Another problem faced by infants receiving neonatal intensive care is psycho-emotional. Physical and emotional closeness are crucial to the physical, emotional and social well-being of both the infants and their parents. Infants needing neonatal intensive care show impairment in neurophysiological organization, perceptual-cognitive development and motor development. Their parents suffer from impaired parental bonding, moods, perceptions, interactive behavior with their baby and increased parental stress.

Kangaroo care, wherein the neonate is strapped onto the chest of one of the parents, allowing for skin-to-skin contact, has been shown to drastically reduce neonatal morbidity and mortality, while increasing bonding with the parents and allowing for a better neurocognitive development of the infant.

However, kangaroo care is often not possible. E.g., when the infant requires ventilator support, which is often the case with preterm infants; or in case an infant requires phototherapy for neonatal jaundice.

Further, the preterm infant is generally very tiny and fragile, making it very difficult for the parent to safely handle the infant during kangaroo care, without injuring the infant's skin or damaging its neck/limbs.

There is thus a need for a means to provide infant intensive care in an easy-to-use manner that does not require skilled medical staff, and which can simultaneously provide the physical, emotional and psychological health benefits of kangaroo care for both, infant and parents.

### SUMMARY OF INVENTION

Embodiments of the present invention are directed to infant care apparatuses and systems that provide easy-to-operate intensive and ambulatory infant care comprising: a molded tray to carry the infant, said molded tray being molded in the shape of an infant's body for convenient and safe handling of the infant without risk of damaging the infant's neck and limbs; and one or more fastening devices to fasten the molded tray around the caregiver's chest; in a manner allowing skin-to-skin contact of the front of the infant's body with the caregiver's chest.

In some embodiments the infant care apparatus and system may comprise additionally a plurality of sensors to monitor various physiological parameters of the infant such as body temperature, heart rate, blood pressure, respiratory rate and oxygenation levels.

In some embodiments, the infant care apparatus and systems may comprise additionally a negative pressure ventilator, said negative pressure ventilator comprising a cuirass shell that encloses at least a part of the infant's thoraco-abdominal area; said cuirass shell, along with the molded tray of claim 1, forming an airtight seal around that part of the infant's thoraco-abdominal area; a ventilation unit with one or more pumps to vary the pressure around the infant's enclosed thoraco-abdominal area to alternately initiate inspiration and expiration, to provide safe mechanical ventilation to the infant even during kangaroo care; and a plurality of sensors to regulate the functioning of the ventilator, such as pressure sensors, blood oxygenation sensors and breath sensors. In some embodiments the ventilator may comprise additionally a burp mode setting to induce the infant to burp, by providing one or more high pressure compressions during expiration.

In some embodiments, the infant care apparatus and systems may comprise additionally a nappy casing shell around the infant's pelvis; one or more sensors to detect when the infant has passed urine or feces; a plurality of disposable bags for collection of the infant's urine and feces; one or more devices to dispense and seal the disposable bags; a pipe that allows for at least one of the following: suctioning of the urine and feces, fine mist or bubbles to wash the infant's skin, and dry air to dry the infant's skin; and an infant cleaning system comprising at least one of the following; vacuum pump for suctioning the urine and feces, warm mist generator for washing the infant, and compressor for dry air to dry the infant after washing.

In some embodiments, the infant care apparatus and systems may comprise additionally one or more heart activity detection sensors that monitor the infant's cardiac activity and provide feedback to the ventilation unit, to pump air at high pressures when required, at a suitable rate, to perform external cardiac compressions to stimulate the infant's heart in the case of a cardiac arrest; and/or a heat pump to provide the desired temperature to the infant in a safe manner; and/or a plurality of light emitting diodes to provide phototherapy as required by the infant; and/or an aspirator for suctioning secretions from nose and/or mouth of infant; and/or one or more temperature sensors and at least one of: warm mist generating apparatus to humidify the air around the infant to prevent evaporative losses, and a heat pump to provide the desired temperature to the infant in a safe manner; and/or one or more alarms designed not to disturb the infant by alerting the caretakers by one or more of the following means: vibrations, light, text messages and voice messages; and/or a speaker to play recorded messages of the mother's heart beat to soothe the infant even when the infant is away from the mother.

In some embodiments all the components of the said apparatus and system are molded together, to form one unit that can be used in one of two ways: strapped on to the caregiver; and placed on a horizontal surface in case kangaroo care is not possible.

The principal object of this invention is to provide infant care apparatuses and systems that allow for safe and easy to manage kangaroo care, along with intensive care and monitoring of the infant, without requiring a skilled operator.

Another object of this invention is to make an infant care apparatus and system that can be remotely controlled by doctors/health care providers, to stabilize the infant's physiological parameters.

Another object of this invention is to provide safe mechanical ventilation as part of the infant care apparatus and system.

Another object of this invention is to provide external cardiac compressions to the infant, when required.

Another object of this invention is to help in burping the infant.

Another object of this invention is to help in aspiration of the infant's oral secretions and gastric contents.

Another object of this invention is to provide automated urine and feces collection, disposal and cleaning of the infant, which may also include automated measuring and testing to assess the infant's health status.

Another object of this invention is to be able to securely fit infants of slightly different sizes in the same molded tray.

Another object of this invention is to prevent evaporative heat losses of the infant.
Another object of this invention is to better understand the infant's neurophysiological status, to enable the caregiver to pre-emptively solve the infant's problems and increase its comfort and ease, by providing an electroencephalograph (EEG) cap as part of the invention.

Another object of this invention is to provide phototherapy to the infant if required.

Another object of this invention is to maintain the desired body temperature of the infant in a safe manner.

Another object of this invention is to provide alarms that are gentle and will not stress the infant.

Another object of this invention is to make the infant feel as if it is close to the mother, even if kangaroo care is not possible.

While the invention is described herein by way of example using several embodiments and illustrative drawings, those skilled in the art will recognize that the invention is not limited to the embodiments of drawing or drawings described, and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modification, equivalents and alternatives falling within the spirit and scope of the present invention as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include,” “including,” and “includes” mean including, but not limited to. Further, the words “a” or “an” mean “at least one” and the word “plurality” means one or more, unless otherwise mentioned. Various objects, features, aspects, and advantages of the present invention will become more apparent from the following detailed description of preferred embodiments of the invention, along with the accompanying drawings in which like numerals represent like components.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

These and other features, benefits and advantages of the present invention will become apparent by reference to the following text figures, with like reference numbers referring to like structures across the views, wherein:

FIG. 1 illustrates an outline view of the molded tray according to one embodiment of the present invention, fastened on a caregiver.

FIG. 2 illustrates a perspective view of the molded tray according to one embodiment of the present invention.

FIG. 3 illustrates a perspective view of the molded tray along with mattress and heat pump according to one embodiment of the present invention.

FIG. 4 illustrates a perspective view of the molded tray along with negative pressure ventilator according to one embodiment of the present invention.

FIG. 5 illustrates a perspective view of the urine and feces disposal system and aspirator according to one embodiment of the present invention.

FIG. 6 illustrates a perspective view of the infant care apparatus and system according to one embodiment of the present invention.

### DETAILED DESCRIPTION OF THE INVENTION

The present invention discloses infant care apparatuses and systems directed towards providing safe infant handling and kangaroo type intensive care to infants, greatly reducing the infant morbidity and mortality rates, while at the same time greatly increasing infant and maternal comfort and ease. Various embodiments of the invention are described below.

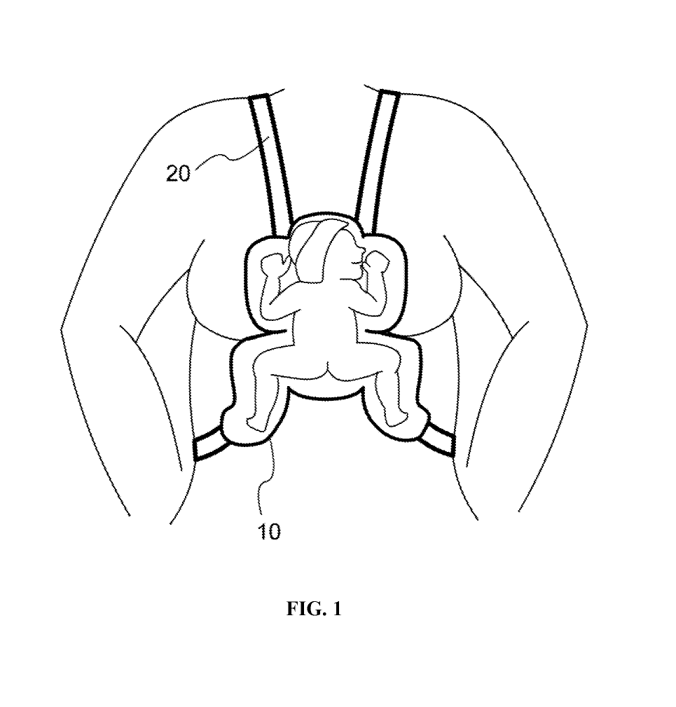

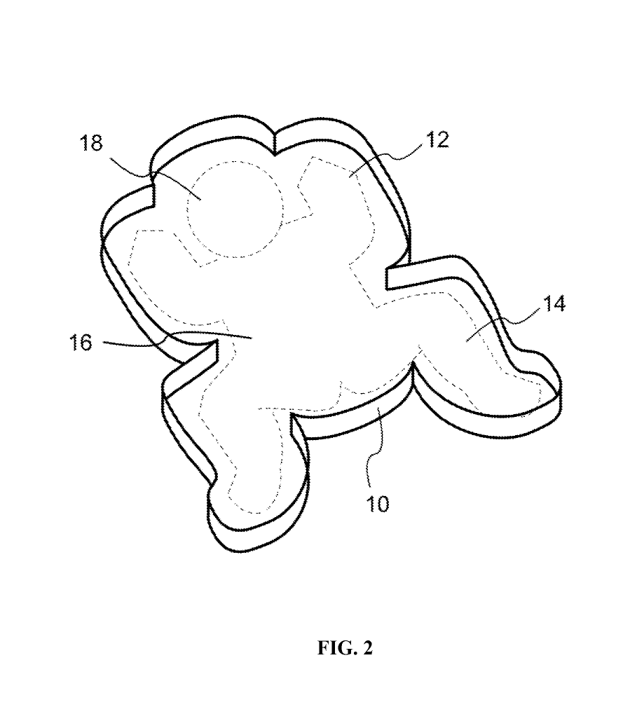

FIGS. 1 and 2 illustrate different views of one embodiment of the invention.

FIG. 1 illustrates an outline view of the infant care apparatus and system according to one embodiment of the invention. FIG. 2 illustrates a perspective view of the infant care and monitoring apparatus of the same embodiment. The infant is placed in molded tray 10, which is shaped with separate depressions 12 for the upper limbs, separate depressions 14 for the lower limbs, depression 16 for the thorax and abdomen and depression 18 for the head and neck. The molded tray 10 is fastened with straps 20 which could go around the caregiver's waist and could even allow the tray to hinge at the caregiver's waist. The straps could also go over the shoulders of the caregiver, such that the infant placed within the molded tray 10 is in skin-to-skin contact with the chest of the caregiver. This allows for kangaroo care of the infant. The molded tray, shaped to support different parts of the body, allows for safe and easy handling of the infant.

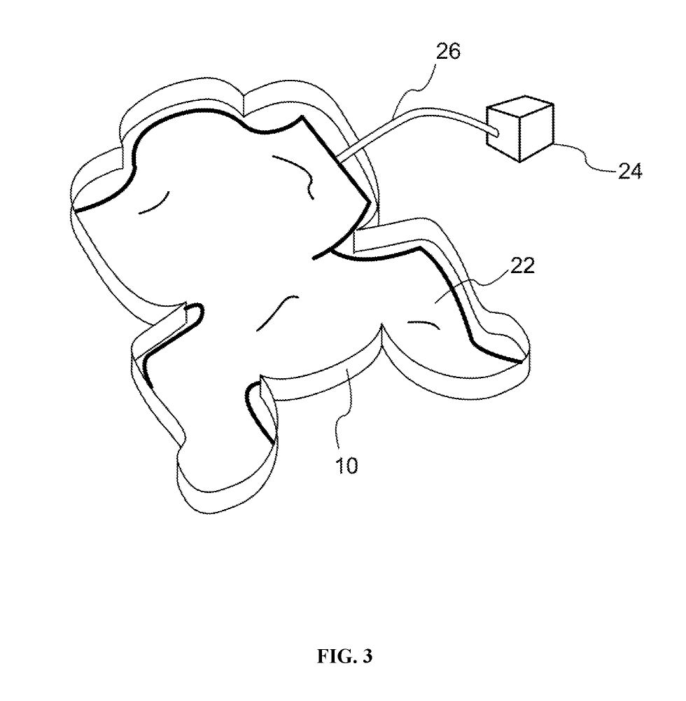

FIG. 3 illustrates a perspective view of the one of the embodiments of the invention, further showing a mattress 22 placed in the molded tray 10, which allows for snug fit of the infant in the molded tray 10. There is a heat pump 24 which can be strapped to the caregiver's waist, or which can be fixed to the molded tray 10 itself, which provides heating/cooling as required to the infant via tube 26 connected to mattress 22. Heated/cooled air is pumped via tube 26 by heat pump 24 to mattress 22, to provide gentle heating/cooling as required to maintain desired body temperature of the infant.

Kangaroo care is very beneficial method of providing special infant care. The close proximity of the infant's skin with the caregiver's skin helps naturally maintain body temperature of the infant to the optimum temperature, without the risk of burning the infant, and without the use of electrical power.

Further, skin-to-skin contact provided by kangaroo care helps improve parental bonding with the infant and reduces stress for both the infant and the caregiver, who is usually one of the parents.

Further, an infant under kangaroo-care is under constant surveillance by the caregiver, and can be immediately attended to in the case of any health issue.

The molded tray 10 of this embodiment of the invention is very useful in providing convenient and easy to handle kangaroo care, as all parts of the infant's body—upper limbs, lower limbs, thorax and abdomen, and head and neck are all well supported in separate depressions. The depressions 14 for the lower limbs and the depressions 16 for the upper limbs are shaped to allow the infants limbs to be placed laterally on both sides of the body, with lower limbs bent at the knees and upper limbs bent at the elbow, as illustrated in FIG. 1, which is a comfortable position for the infant. The tray could also be shaped differently, or flexible to support other positions of the infant. The molded tray 10 of this embodiment is especially useful for preterm infants, who require special care. Their bodies are very tiny and skin and bones are fragile and can easily get injured during handling, and such a shaped tray would make handling such infants easy and convenient.

The molded tray 10 can be preferably made of thermoformed plastic, silicone rubber, fiberglass, or any other material that can form lightweight trays. It can be made in various sizes, to suit different sized infants. If the molded tray 10 is made of stiff material, it can preferably have a soft mattress 22 placed between it and the infant, to allow for a snug, comfortable fit, even for infants of different sizes.

The mattress 22 is preferably made of a low spring-back material, like memory foam, or a low spring-back gel, or is made of a stretchable fabric cover filled with beads. This allows for the mattress to conform to the infant's body, allowing for movement of the infant's body parts in the separate depressions 12, 14, 16 and 18, while still snugly securing the infant in the molded tray 10.

In other embodiments, the mattress 22, or the molded tray 10 could have light emitting diodes, to provide suitable phototherapy to the infant, if required.

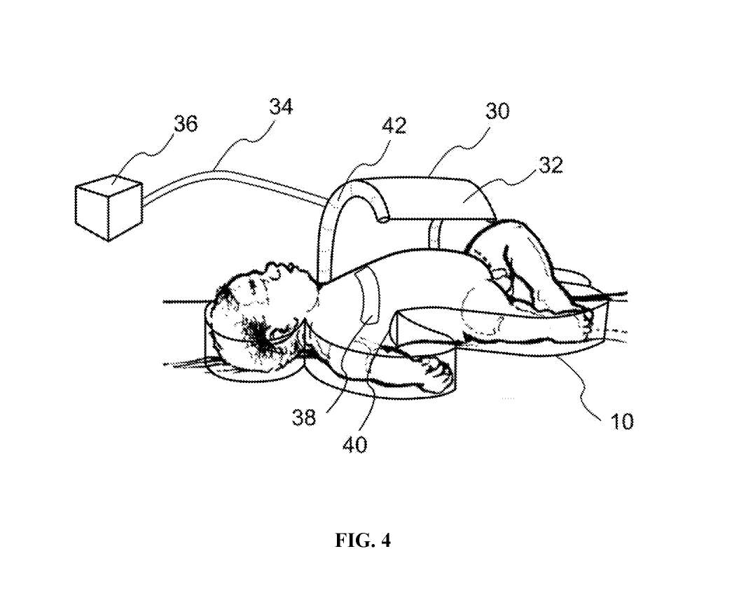

FIG. 4 illustrates another embodiment of the said invention, which additionally comprises a negative pressure ventilator as part of the infant care and management apparatus and system. Negative pressure ventilator 30 comprises cuirass shell 32 enclosing the infant's thoraco-abdominal area, ventilation pipe 34, ventilation unit 36 comprising pumps, valves and sensors. Ventilation unit 36 can be strapped around the caregiver's waist, or placed on the bed or can even be part of the molded tray 40. Further, this embodiment has electrocardiogram (ECG) sensors 38 fixed to the infant's chest. The cuirass shell 32 forms an airtight seal around the infant's thoraco-abdomen, along with molded tray 10. The sensors on the ventilation unit 36 detect the infant's respiratory parameters and initiate respiratory support as and when required, to stabilize the infant's blood oxygen levels. The pumps in the ventilation unit alternately vary the air pressure in the area enclosed by the cuirass shell, creating a vacuum to initiate inspiration or increasing the pressure to initiate expiration. The negative pressure ventilator 30 is advantageous as it is non-invasive, and allows for instant starting of ventilation the moment you close the cuirass shell 32 over molded tray 10, if required. Similarly, the electrocardiogram (ECG) sensors 38 send signals to the ventilation unit 36 to start external cardiac compressions in the case of a cardiac arrest. The ventilation unit 36 can also perform external cardiac compressions by alternately varying the pressure around the enclosed thoraco-abdominal cavity at levels higher than those required to initiate respiration.

The ventilation unit 36 in addition to providing ventilation and external cardiac compression could also be used to make the infant burp by increasing the expiratory compression a few times. It can even be used to make the infant cough out phlegm by strong inspiration followed by sudden forceful expiration.

FIG. 4 also illustrates inflatable air seals for the negative pressure ventilator. In order to perform negative pressure ventilation, a portion of the infant's thoraco-abdominal cavity has to be enclosed within a cuirass shell with an airtight seal. This allows the ventilation unit to generate alternately generate vacuum and high pressure around the enclosed thoraco-abdominal area, allowing initiation of inspiration and expiration. In FIG. 4, inflatable air seals 40 are visible on the molded tray 10 and inflatable air seals 42 are visible on the undersurface of cuirass shell 32. Both these air seals can be inflated by the pumps in the ventilation unit 36. By having inflatable air seals, infants of different body sizes can be accommodated with an airtight seal in the same molded tray 10.

In other embodiments, the air seal could be made of soft gel like material or even of a stretchable material filled with beads that could mold itself around the infant's body.

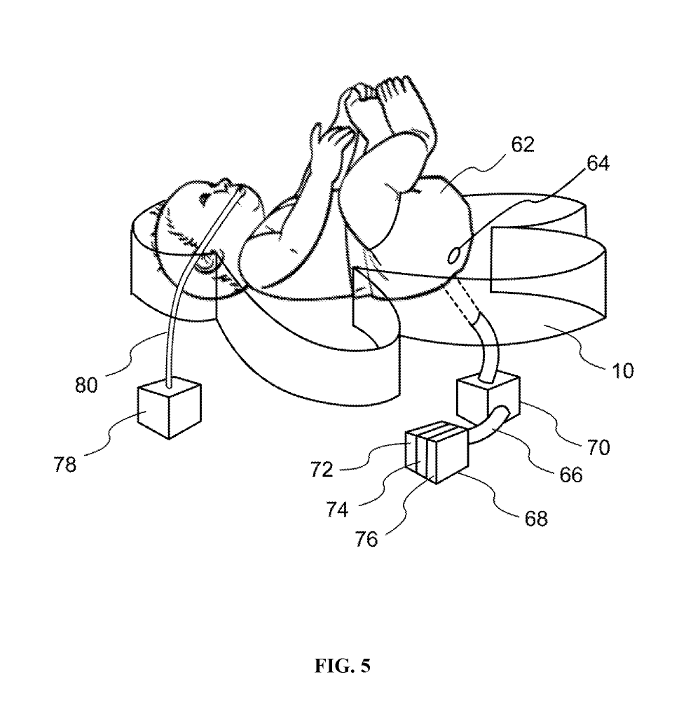

FIG. 5 illustrates another embodiment of this invention which has, in addition to molded tray 10, a nappy casing shell 62 around the infant's pelvis with sensors 64 to detect whether the infant has passed urine or feces. The nappy casing shell 62 could be separate or part of the molded tray and is connected via pipe 66 to infant cleaning system 68 via disposable bags dispenser 70. Infant cleaning system 68 consists of vacuum pump 72 for suctioning the urine and feces of the infant, warm mist generator 74 for washing the infant and compressor 76 with heater for warm dry air to dry the infant after washing. The pipe 66 allows for suctioning of the urine and feces, passage of fine mist or bubbles to wash the infant's skin and dry air to dry the infant's skin by the infant cleaning system 68. In some embodiments the invention could include automated measuring and testing of the urine and/or feces, to assess the infant's health status.

Another pump 78 can be used to suction out oral secretions and gastric contents if required, via pipe 80 that can be inserted into the infant's nose and/or mouth for suctioning, as required.

Warm mist generator 74 can also be diverted via a valve or another warm mist generator can be used to increase the humidity around the infant, to prevent evaporative losses, which are a major cause of hypothermia especially in the premature infant.

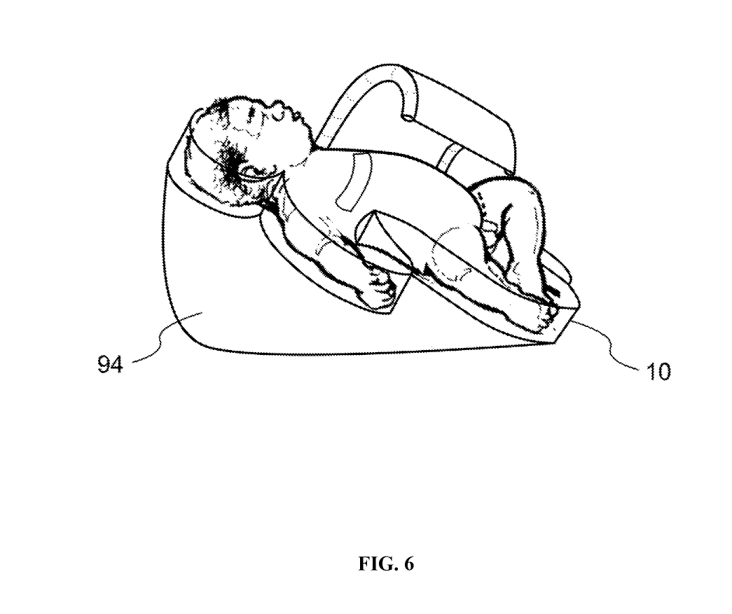

FIG. 6 illustrates an embodiment of the invention, wherein molded around the base of molded tray 10, is the infant monitoring and stabilization unit 94. Infant monitoring and stabilization unit 94 comprises all the parts of the invention other than the molded tray 10—the sensors, pumps, battery, electronics, power supply, etc. used to monitor and stabilize the infant's vital parameters, clean the infant, humidify it, etc., making the infant care and monitoring apparatus and system a one piece lightweight unit that can be strapped across the caregiver's chest.

The infant monitoring and stabilization unit 94 can also act as a stand, to sturdily prop the molding tray 10 when placed on a horizontal surface like a bed/table.

In some embodiments, the signals from the sensors in infant monitoring and stabilization unit 94 can be relayed to doctors/healthcare providers at other geographical locations, and they can in turn remotely, program the various parts of the infant monitoring and stabilization unit 94. E.g. the respiratory rate or pressures on the ventilator, or the desired temperature for the heat pump, etc. Alarms from the device can also instantly alert the doctors/healthcare providers via phone calls/messages, so that they can immediately attend to the infant and change any device parameters as required to solve the problem.

In some embodiments, the alarms on the device, e.g. for detecting low oxygen levels, low temperature, etc. can be gentle, not alarming to the infant in the form of voice messages from a speaker on the device, or lights flashing, or messages and phone calls to the caregiver/healthcare providers, or even vibrations that can be felt by the caregiver when the infant care and monitoring apparatus and system is strapped across the caregiver's chest.

In other embodiments, the infant monitoring and stabilization unit could be strapped onto the waist of the caregiver, or worn on the caregiver's back.

In some embodiments, kangaroo care might not be possible. In such cases, the infant can still be placed in this infant care and monitoring apparatus and system, on a horizontal surface like a bed/table/floor. The infant can still get some of the benefits of kangaroo care by having recorded messages of the infant's mother's heartbeat being played to the infant by the infant care and monitoring apparatus and system.

In some embodiments the infant care apparatus and system may include an electroencephalograph (EEG) cap, to better understand the infant's neurophysiological status and to enable the caregiver to pre-emptively solve the infant's problems and increase its comfort and ease.

In some embodiments the infant care apparatus and system may include a networking module that can transmit data from the plurality of sensors to one or more remote servers from where the data can be accessed by medical professionals; and can receive back operating commands from the one or more remote servers, via any wired or wireless network. This gives it the advantage of immediate skilled intervention to provide intensive care, even in locations where no skilled medical staff is available.

In the foregoing specification, specific embodiments of the present invention have been described. However, one of ordinary skill in the art will appreciate that various modifications and changes can be made without departing from the spirit and scope of the present invention as set forth in the various embodiments discussed above and the claims that follow. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements as described herein.

We claim:

1. An infant care apparatus comprising:
a tray (10) that is molded in a shape of an infant's body to carry the infant, wherein the molded tray (10) comprises,
a plurality of depressions (12-18) for accommodating different parts of the infant's body; and
a nappy casing shell that surrounds the infant's pelvis, wherein the nappy casing shell (62) comprises,
at least one sensor (64) that detects a presence of urine or feces inside the nappy casing shell (62) when the infant has passed the urine or feces;
a plurality of disposable means (70) for collection of the infant's urine and feces; and
at least one device that seals and dispenses the disposable means that collects the infant's urine and feces;
an infant cleaning system (68) comprising at least one;
a vacuum pump (72) for suctioning the urine and feces when the sensor (64) detects the presence of urine or feces inside the nappy casing shell (62);
a warm mist generator (74) that generates warm mist or fine bubbles for washing the infant's skin after the vacuum pump (72) suctions the urine or feces from the nappy casing shell (62); and
a compressor (76) that generates dry air for drying the infant's skin after the warm mist generator (74) washes the skin of the infant;
a conduit (66) that connects the molded tray (10) with the infant cleaning system (68) through disposable means dispenser (70), wherein the conduit allows at least one of:
suctions urine and feces when the sensor (64) detects the urine or feces of the infant;
fine mist or bubbles to wash the infant's skin after the vacuum pump (72) suctions the urine or feces of the infant; and
dry air to dry the infant's skin after the warm mist generator (74) washes the infant's skin by fine mist or bubbles; and
one or more fastening devices (20) to fasten the molded tray (10) around the caregiver's chest in a manner allowing skin-to-skin contact of the front of the infant's body with the caregiver's chest.

2. The infant care apparatus of claim 1, comprising additionally: a plurality of sensors to monitor various physiological parameters of the infant.

3. The infant care apparatus of claim 2, comprising additionally: a networking module that is communicatively coupled to the plurality of sensors, wherein the networking module is configured to receive and transmit sensor data comprising the physiological condition of the infant from the plurality of sensors to one or more remote servers, wherein the one or more servers provide the sensor data to medical professionals; and is configured to receive commands from the medical professional through a wired or a wireless network.

4. The infant care apparatus of claim 1, comprising additionally:
a negative pressure ventilator (30), wherein said negative pressure ventilator (30) comprises:
a cuirass shell (32) that encloses at least a part of the infant's thoraco-abdominal area wherein said cuirass shell (32), along with the molded tray (10) of claim 1, forming an airtight seal around that part of the infant's thoraco-abdominal area;
a ventilation unit (36) that includes one or more pumps to vary the pressure around the infant's thoraco-abdominal area to alternately initiate inspiration and expiration; and
a plurality of sensors configured to monitor a physiological condition of the infant and to regulate the functioning of the ventilator based on the monitored physiological condition of the infant, wherein said plurality of sensors includes at least one of pressure sensors, blood oxygenation sensors or breath sensors.

5. The infant care apparatus of claim 4, wherein the ventilation unit (36) comprising additionally:
a burp mode setting unit to induce the infant to burp, by providing one or more high pressure compressions during expiration.

6. The infant care apparatus of claim 4, comprising additionally:
at least one heart activity detection sensor (38) that is configured to monitor the infant's cardiac activity and transmit the monitored heart activity of the infant's to the ventilation unit (36), to pump air at high pressures at a suitable rate to perform external cardiac compressions to stimulate the infant's heart when cardiac arrest of the infant is detected.

7. The infant care apparatus of claim 1, comprising additionally:
a plurality of light emitting diodes that provides phototherapy as required by the infant, said plurality of light emitting diodes being placed in any one of:
the molded tray (10); and
a mattress (22) placed in the molded tray (10).

8. The infant care apparatus of claim 1, wherein:
the molded tray (10) has a lining that is made of a low spring-back material which can snugly mold around the infant's body, further securing the infant in the tray (10).

9. The infant care apparatus of claim 1, comprising additionally:
an aspirator (78) for suctioning secretions from nose and/or mouth of infant.

10. The infant care apparatus of claim 1, comprising additionally:
one or more temperature sensors; and at least one of
warm mist generating apparatus (74) to humidify the air around the infant to prevent evaporative heat losses; and
a heat pump (24) to provide the desired temperature to the infant in a safe manner.

11. The infant care apparatus of claim 1, comprising additionally:
one or more alarms designed not to disturb the infant by alerting the caretakers by one or more of the following means: vibrations, light, text messages and voice messages.

12. The infant care apparatus of claim 1, comprising additionally:
a speaker to play recorded messages of the mother's heart beat to soothe the infant even when the infant is away from the mother.

13. The infant care apparatus of claim 1, wherein all the components of the said apparatus and system are molded together, to form one unit that can be used in one of two ways:
strapped on to the caregiver; and
placed on a horizontal surface in case kangaroo care is not possible.

14. The infant care apparatus of claim 1, comprising additionally:
an electroencephalograph cap configured to analyse the infant's neurophysiological status, to enable the caregiver to pre-emptively solve the infant's problems and increase its comfort and ease.
